(19) United States
(12) Patent Application Publication
(10) Pub. No.: US 2024/0145042 A1
(43) Pub. Date: May 2, 2024

(54) MEDICAL INFORMATION PROCESSING DEVICE, MEDICAL INFORMATION PROCESSING METHOD, AND STORAGE MEDIUM

(71) Applicant: CANON MEDICAL SYSTEMS CORPORATION, Otawara-shi (JP)

(72) Inventor: Kazumasa NORO, Shioya (JP)

(73) Assignee: CANON MEDICAL SYSTEMS CORPORATION, Otawara-shi (JP)

(21) Appl. No.: 18/496,998

(22) Filed: Oct. 30, 2023

(30) Foreign Application Priority Data
Nov. 2, 2022 (JP) 2022-176431
Oct. 25, 2023 (JP) 2023-183174

(51) Int. Cl. G16H 10/00; G16H 50/20

(52) U.S. Cl. CPC G16H 10/00; G16H 50/20

(57) ABSTRACT

According to an embodiment, a medical information processing device includes processing circuitry. The processing circuitry acquires text data of a processing target and outputs specific information for identifying one item of medical information satisfying a specific condition regarding a diagnosis target based on a medical term included in the text data and a medical ontology in which a relationship between two or more items of the medical information is defined.

### CROSS-REFERENCE TO RELATED APPLICATION

The present application claims priority based on Japanese Patent Application No. 2022-176431, filed Nov. 2, 2022, and Japanese Patent Application No. 2023-183174, filed Oct. 25, 2023, the content of which is incorporated herein by reference.

### FIELD

Embodiments and drawings disclosed herein relate to a medical information processing device, a medical information processing method, and a storage medium.

### BACKGROUND

In recent years, the composition of medical data related to medical care has become more complex. For example, information such as discharge summaries is structured and recorded according to a subjective, objective, assessment, and plan (SOAP) format in the medical field, but it is difficult to organize and record a problem list for each disease of hospitalized patients. For this reason, there is a growing demand for a method of organizing increasingly complex medical data.

In this regard, technology for clustering text of medical data according to the subject and extracting a concept of each generated cluster has been conventionally proposed. However, in conventional technology, it is difficult to describe a relationship between words included in medical data. Furthermore, no technology related to identifying important words in medical data or grouping similar words has been proposed so far. For this reason, it is considered difficult to organize extracted information and identify important information when a huge number of concepts and relationships between concepts are extracted from medical data with the technology proposed so far.

### BRIEF DESCRIPTION OF THE DRAWINGS

FIG. 1 is a diagram showing an example of a functional configuration and usage environment of a medical information processing device according to an embodiment.

FIG. 2 is a diagram schematically showing an example of medical ontology used by the medical information processing device according to the embodiment.

FIG. 3 is a diagram schematically showing an example of a mapping process of a mapping processing function provided in the medical information processing device according to the embodiment.

FIG. 4 is a diagram schematically showing another example of the mapping process of the mapping processing function provided in the medical information processing device according to the embodiment.

FIG. 5 is a diagram schematically showing an example of a first identification process of an identification function provided in the medical information processing device according to the embodiment.

FIG. 6 is a diagram schematically showing an example of a second identification process of the identification function provided in the medical information processing device according to the embodiment.

FIG. 7 is a diagram schematically showing an example of a third identification process of the identification function provided in the medical information processing device according to the embodiment.

FIG. 8 is a diagram schematically showing an example of a fourth identification process of the identification function provided in the medical information processing device according to the embodiment.

FIG. 9 is a diagram schematically showing an example of a fifth identification process of the identification function provided in the medical information processing device according to the embodiment.

FIG. 10 is a diagram showing an example of a display screen on which the medical information processing device according to the embodiment provides information.

FIG. 11 is a diagram schematically showing an example of a modified example of the fifth identification process of the identification function provided in the medical information processing device according to the embodiment.

FIG. 12 is a diagram showing another example of a display screen on which the medical information processing device according to the embodiment provides information.

FIG. 13 is a flowchart showing an example of a flow of a process of the medical information processing device according to the embodiment.

### DETAILED DESCRIPTION

According to an embodiment, a medical information processing device includes processing circuitry. The processing circuitry acquires text data of a processing target and outputs specific information for identifying one item of medical information satisfying a specific condition regarding a diagnosis target based on a medical term included in the text data and a medical ontology in which a relationship between two or more items of the medical information is defined.

Hereinafter, a medical information processing device, a medical information processing method, and a storage medium of embodiments will be described with reference to the drawings. The medical information processing device is, for example, a device for supporting a diagnosis process of a physician by organizing medical data that is a result of diagnosing a patient at a medical institution where hospital information systems (HIS) have been introduced. The medical information processing device organizes the importance of the information included in the medical data so that it is easy to understand and present the organized importance to the physician. The medical information processing device, for example, organizes information stored in a server device or storage device incorporated in a medical institution network, a cloud computing system, or the like and presents the organized information to the physician.

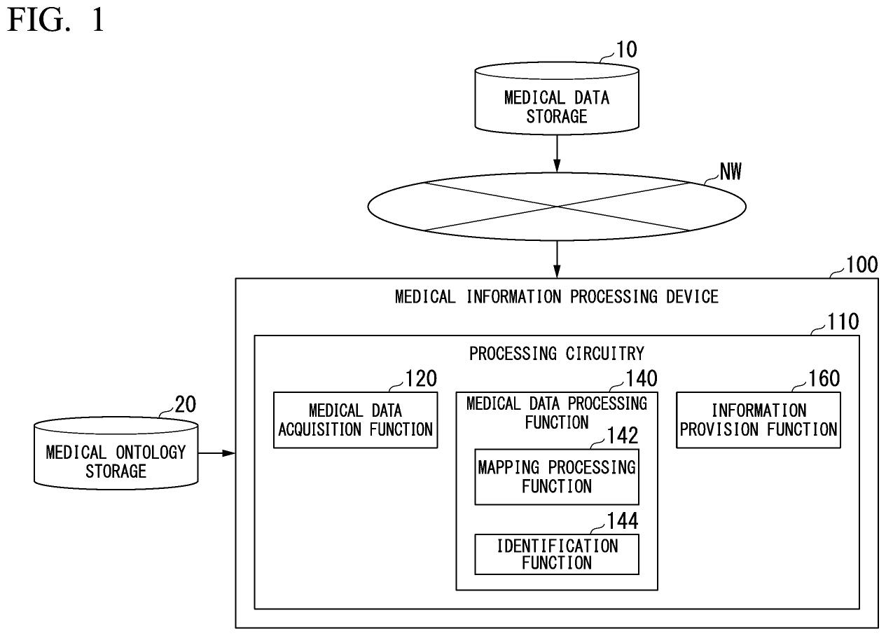

FIG. 1 is a diagram showing an example of a functional configuration and usage environment of the medical information processing device according to the embodiment. The medical information processing device 100 communicates, for example, with a medical data storage 10, which is a storage device that stores various information about the medical treatment of the patient, via a network NW. The network includes, for example, the Internet, a wide area network (WAN), a local area network (LAN), a provider device, a radio base station, and the like. The medical information processing device 100 may be implemented by a server device on a network or the like.

The medical data storage 10 stores information (medical data) about examinations and diagnoses performed on the patient. The medical data storage 10 is, for example, a storage device that stores data such as an examination result when the patient is examined and a medical record (an electronic medical record) when the patient is treated. An electronic medical record includes at least a sentence representing information about the medical treatment of the patient is represented in text. In the following description, it is assumed that the electronic medical record data is stored in the medical data storage 10 and the medical information processing device 100 processes medical information included in the electronic medical record of a patient who is a current diagnosis target (hereinafter referred to as a “diagnosis target patient”) as medical data. The medical data is an example of a “processing target.” The diagnosis target patient is an example of a “diagnosis target.”

The medical information processing device 100 extracts a medical term related to the medical treatment of the diagnosis target patient from the sentence of the text included in the medical data and maps the extracted medical term to a medical ontology stored in the medical ontology storage 20. Medical terms include medical-treatment-related words (hereinafter referred to as “medical words”), time information such as periods and times related to the medical treatment, and the like. The medical terms may include information for identifying the diagnosis target patient, such as a name of the diagnosis target patient (a patient name). The medical information processing device 100 performs a clustering process on the basis of the medical ontology in which medical terms are mapped and identifies medical terms (for example, disease names and the like) that are of high importance for a disease that the diagnosis target patient is suffering from.

The medical ontology storage 20 is a storage device that stores the configuration of a basic medical ontology. The medical ontology is a structural framework in which the relationships between medical information in the medical field, such as various information and events related to diseases, are defined. In the medical ontology, for example, in relation to the name of the disease, relationships between items of various medical information such as symptoms of the disease, a site where the symptom occurs, a corresponding therapeutic method, examination items useful for diagnosis, an examination result (an examination value) indicating that the patient is determined to be suffering from the disease, and therapeutic drugs (prescription drugs) corresponding to the disease are shown.

Although a configuration in which the medical ontology storage 20 is connected to the medical information processing device 100 in FIG. 1 is shown, the medical ontology storage 20 may be a storage device built into the medical information processing device 100 or may be a storage device connected to the network NW and accessed by the medical information processing device 100 via the network NW. When the medical ontology storage 20 is a storage device connected to the network NW, the medical ontology storage 20 and the medical data storage 10 are the same storage device, i.e., may be a configuration in which the medical ontology stored in the medical ontology storage 20 is stored in the medical data storage 10.

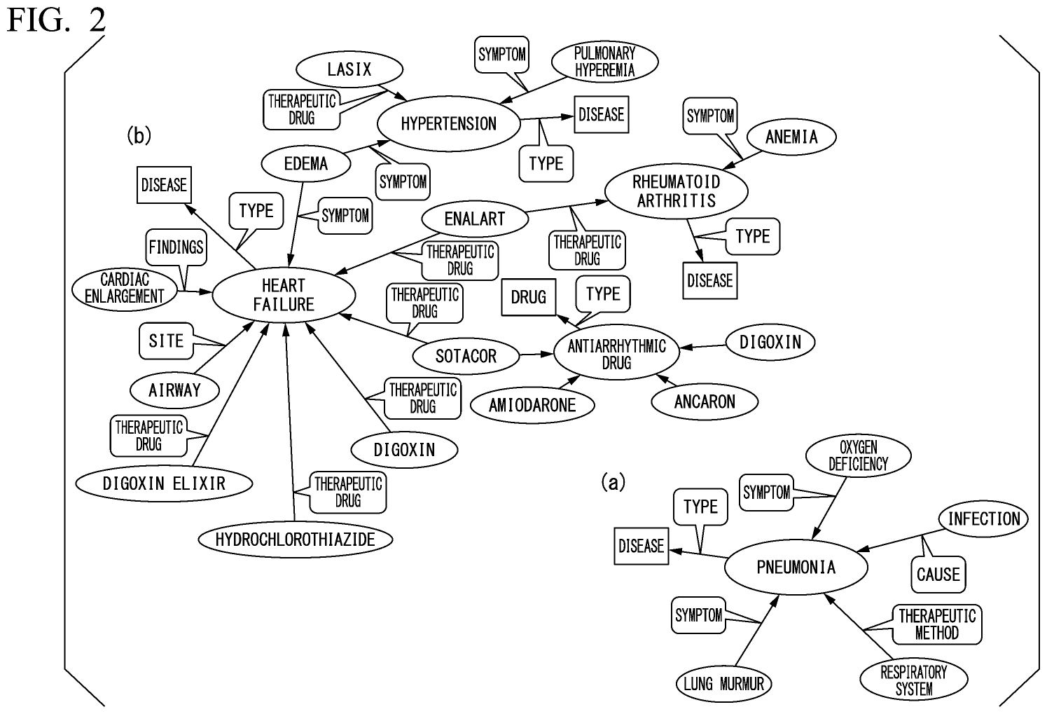

Here, an example of a configuration of a basic medical ontology stored in the medical ontology storage 20 will be described. FIG. 2 is a diagram schematically showing an example of a medical ontology used by the medical information processing device 100 according to the embodiment. In the basic medical ontology structure, a plurality of entities indicating medical information, such as medical words, are associated on the basis of relationships between medical information items. In the entity, for example, medical words indicating a name of a disease, symptoms that can occur due to the disease, a site where the symptoms may occur, a cause of occurrence of the disease, a therapeutic method, a type of therapeutic drug (generic term), a name of the therapeutic drug, and the like are shown as medical information. In the basic medical ontology, for example, one or more lower-level concept entities (hereinafter referred to as “lower-level entities”) that have a relationship are connected (linked) to higher-level concept entities (hereinafter referred to as “higher-level entities”) indicating the name of a disease and a type of therapeutic drug (generic term). Entities (hereinafter referred to as “category entities”) indicating category information indicating a type of medical word indicated in the entity are also connected (linked) to the higher-level entity and the lower-level entity. In the category entity, for example, information for classifying types (categories) of medical words such as diseases, symptoms, severity of symptoms, physician's findings, examinations, sites, causes, therapeutic methods, drugs, and the like is indicated. In FIG. 2(a), an example of a basic medical ontology related to the name of the disease=“pneumonia” is shown. In FIG. 2(b), an example of a basic medical ontology related to the name of the disease=“heart failure” is shown. In FIG. 2, an example in which a category entity is connected only to a higher-level entity, i.e., a category entity connected to a lower-level entity is omitted, is shown.

In the basic medical ontology shown in FIG. 2(a), an example in which the category entity with the medical word category “disease” is connected to the higher-level entity whose disease name is “pneumonia,” lower-level entities of “lung murmur” and “oxygen deficiency” whose relationship is “symptom” are connected, a lower-level entity of “infection” whose relationship is “cause” is connected, and a lower-level entity of “respirator” whose relationship is “therapeutic method” is connected is shown.

In the basic medical ontology shown in FIG. 2(b), an example in which the category entity with the medical word category “disease” is connected to the higher-level entity whose disease name is “heart failure,” the lower-level entity of “edema” whose relationship is “symptom” is connected, the lower-level entity of “cardiac enlargement” whose relationship is “finding” is connected, the lower-level entity of “airway” whose relationship is “site,” and the lower-level entities of “Enalart,” “Sotacor,” “digoxin,” “hydrochlorothiazide,” and “digoxin elixir” whose relationship is “therapeutic drug” are connected is shown. Furthermore, in the basic medical ontology shown in FIG. 2(b), an example in which the category entity with the medical word category “disease” is connected to the higher-level entity whose disease name is “hypertension,” the lower-level entities of “edema” and “pulmonary hypertension” whose relationship is “symptom” are connected, and the lower-level entity of “Lasix” whose relationship is “therapeutic drug” is connected is shown. Furthermore, in the basic medical ontology shown in FIG. 2(b), the category entity with the medical word category “disease” is connected to the higher-level entity whose disease name is “rheumatoid arthritis,” the lower-level entity of “anemia” whose relationship is “symptom” is connected, and the lower-level entity of “Enalart” whose relationship is “therapeutic drug” is connected. Furthermore, in the basic medical ontology shown in FIG. 2(b), the category entity with the medical word category of “drug” is connected to the higher-level entity of the type of therapeutic drug (generic term) of “antiarrhythmic drug” and the lower-level entities representing the names of therapeutic drugs of “Sotacor,” “amiodarone,” “Ancaron,” and “digoxin” having efficacy similar to that of an antiarrhythmic drug are connected. Also, in an example of the basic medical ontology shown in FIG. 2(b), the lower-level entity of “edema” whose relationship is “symptom” is connected to the higher-level entities of “heart failure” and “hypertension,” the lower-level entity of “Enalart” whose relationship is “therapeutic drug” is connected to the higher-level entities of “heart failure” and “rheumatoid arthritis,” and the lower-level entity of “Sotacor” whose relationship is “therapeutic drug” is connected to the higher-level entity of “antiarrhythmic drug.” This is because the symptom of “edema” can occur in both diseases of “heart failure” and “hypertension” and “Enalart” is a drug prescribed for both diseases of “heart failure” and “rheumatoid arthritis.” Furthermore, this is because “Sotacor” is one of a plurality of “antiarrhythmic drugs.”

Thus, in the configuration of a basic medical ontology, a plurality of medical words with different relationships may be connected to a single medical word. In other words, the basic medical ontology may be configured so that the relationship between two or more medical words is indicated by a common medical word. Higher-level entities and lower-level entities (which may include category entities) are examples of “medical information.”

### [Configuration of Medical Information Processing Device]

The medical information processing device 100 includes, for example, processing circuitry 110. The processing circuitry 110 executes, for example, processes such as a medical data acquisition function 120, a medical data processing function 140, an information provision function 160, and the like. The medical data processing function 140, for example, is performed to execute processes such as a mapping processing function 142 or an identification function 144. The processing circuitry 110 implements these functions, for example, when a hardware processor executes a program (software) stored in a memory (not shown). The memory (not shown) is implemented by, for example, a semiconductor memory element such as a read-only memory (ROM), a random-access memory (RAM), or a flash memory, a hard disk drive (HDD), an optical disc, or the like.

The hardware processor is, for example, circuitry such as a central processing unit (CPU), a graphics processing unit (GPU), an application-specific integrated circuit (ASIC), or a programmable logic device (for example, a simple programmable logic device (SPLD), a complex programmable logic device (CPLD), or a field programmable gate array (FPGA)). Instead of storing the program in the memory (not shown), the program may be directly embedded in the circuit of the hardware processor. In this case, the hardware processor implements each function by reading and executing the program embedded in the circuitry. The hardware processor is not limited to being configured as a single circuit and may be configured as one hardware processor by combining a plurality of independent circuits to implement each function. A plurality of components may be integrated into one hardware processor to implement each function. Each function may be implemented by incorporating a plurality of components into one dedicated LSI circuit. Here, the program (software) may be stored in advance in a storage device (a storage device having a non-transitory storage medium) that constitutes a semiconductor memory device such as a ROM, a RAM, or a flash memory or a storage device such as a hard disk drive. Alternatively, the program (software) may be stored in a removable storage medium (a non-transitory storage medium) such as a DVD or CD-ROM and installed in a storage device provided in the medical information processing device 100 when the storage medium is mounted in a drive device provided in the medical information processing device 100. The program (software) may be downloaded in advance from another computer device via the network NW and installed in the storage device provided in the medical information processing device 100. A program (software) installed in the storage device provided in the medical information processing device 100 may be transferred to and executed by processing circuitry provided in the processing circuitry 110.

The medical data acquisition function 120 is performed to acquire medical data stored in the medical data storage 10. More specifically, the medical data acquisition function 120 is performed to acquire data of a sentence of text (hereinafter referred to as “text data”) included in the electronic medical record of the diagnosis target patient stored in the medical data storage 10. The medical data acquisition function 120 is performed to acquire the text data, for example, by controlling a communicator (not shown). The medical data acquisition function 120 is performed to output the acquired text data to the medical data processing function 140. The medical data acquisition function 120 may be performed to acquire the medical data itself stored in the medical data storage 10, extract the text data included in the acquired medical data, and output the extracted text data to the medical data processing function 140. The medical data acquisition function 120 is an example of an “acquirer.”

The medical data processing function 140 is performed to divide the text data output by the medical data acquisition function 120 into words and extract a medical term from among the words obtained in the dividing process. Also, the medical data processing function 140 is performed to read the medical ontology stored in the medical ontology storage 20 and map the extracted medical term to the read medical ontology. The medical data processing function 140 is performed to trace medical terms on the medical ontology in which the medical terms are mapped and adjacency between the medical terms and medical words and identify medical words and medical terms (which may be medical data from which medical terms are extracted) with high importance regarding the disease that the diagnosis target patient is suffering from. The medical data processing function 140 is an example of a “processor.”

The mapping processing function 142 is performed to divide the text data output by the medical data acquisition function 120 into words and extract medical terms from the words obtained in the dividing process. Also, the mapping processing function 142 is performed to read the medical ontology stored in the medical ontology storage 20 and map the extracted medical term to a corresponding entity in the read medical ontology.

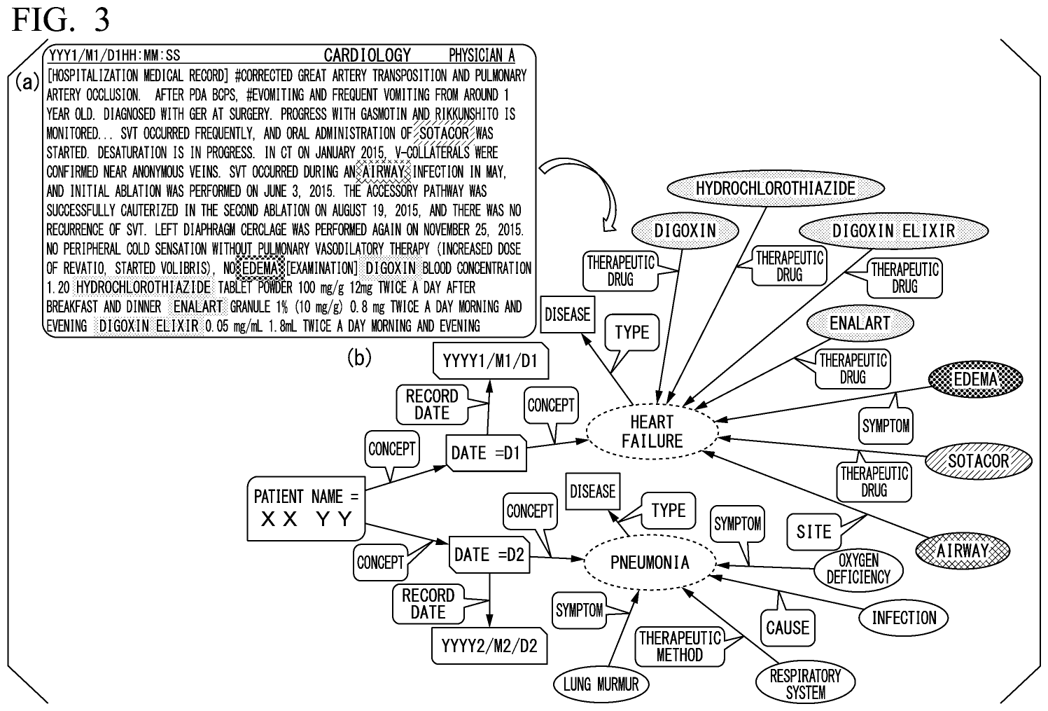

Here, an example of a process in which the mapping processing function 142 is performed to extract a medical term and map the extracted medical term to a medical ontology (hereinafter referred to as a “mapping process”) will be described. FIG. 3 is a diagram schematically showing an example of a mapping process of the mapping processing function 142 provided in the medical information processing device 100 according to the embodiment. In FIG. 3(a), an example of text data output by the medical data acquisition function 120 is shown. In FIG. 3(b), an example in which a medical ontology (hereinafter, referred to as a “mapped ontology”) in which the medical term extracted from the text data in the mapping processing function 142 is mapped is shown.

The mapping processing function 142 is performed to extract a medical term from the text data shown in FIG. 3(a). In FIG. 3(a), an example in which the mapping processing function 142 is performed to extract medical words of “Sotacor,” “airway,” “edema,” “digoxin,” “hydrochlorothiazide,” “Enalart,” and “digoxin elixir” as medical terms is shown.

Also, the mapping processing function 142 is performed to generate a mapped ontology by mapping the extracted medical term to the basic medical ontology configuration as shown in FIG. 2. In FIG. 3(b), an example of a case where the mapping processing function 142 is performed to map medical terms (“Sotacor,” “airway,” “edema,” “digoxin,” “hydrochlorothiazide,” “Enalart,” and “digoxin elixir”) extracted from the text data shown in FIG. 3(a) to the lower-level entity connected to the medical ontology related to “heart failure” shown in FIG. 2(b) is shown. Here, in FIG. 3(b), the lower-level entity of “cardiac enlargement” connected to the medical ontology related to “heart failure” shown in FIG. 2(b) is not mapped because a corresponding medical term is not extracted from the text data shown in FIG. 3(a). Further, in FIG. 3(b), an example of a case where the mapping processing function 142 is performed to map medical terms (here, “lung murmur,” “oxygen deficiency,” “infection,” and “respiratory system”) extracted from text data (not shown) to the lower-level entity connected to the medical ontology related to “pneumonia” shown in FIG. 2(a) is shown.

Although the extracted medical term is only a medical term corresponding to a lower-level entity constituting a basic medical ontology in the example shown in FIG. 3, the extracted medical term may also include a medical term corresponding to a higher-level entity constituting the basic medical ontology. In this case, the mapping processing function 142 is performed to map the extracted medical term to the higher-level entity. Also, the mapping processing function 142 is performed to generate a medical ontology of a configuration including an entity mapped to a medical term and a higher-level entity connected to the entity mapped to the medical term as a mapped ontology.

Here, the mapping processing function 142 may cause the mapped ontology to be generated to have time information. In the example shown in FIG. 3(b), an example of a case where “date” that is a date on which the text data from which the medical term was extracted in the mapping processing function 142 was recorded (described) is connected (linked) and mapped as an entity indicating information of a time concept (hereinafter referred to as an “information entity”) is shown. Here, an information entity of “year, month, and day” indicating information of a record date is connected to the information entity of “date.” Further, the mapping processing function 142 may be performed to indicate a relationship between different entities to which medical terms are mapped. In the example shown in FIG. 3(b), an example of a case where “patient name” indicating the name of a diagnosis target patient who is the target of text data from which the medical term has been extracted in the mapping processing function 142 is connected and mapped as an information entity indicating concept information for associating the higher-level entity of “heart failure” and the higher-level entity of “pneumonia” is shown. Thus, the mapping processing function 142 can be performed to indicate that the higher-level entity of “heart failure” and the higher-level entity of “pneumonia” based on text data recorded on different dates are higher-level entities related to the same diagnosis target patient by connecting information entities indicating various information.

The information entity connected by the mapping processing function 142 may be extracted on the basis of words other than medical words included in the text data (which may be the medical data (electronic medical records) itself) or may be designated, for example, when a primary care physician who diagnoses the diagnosis target patient operates an input interface (not shown) provided in the medical information processing device 100. The input interface is implemented, for example, by a mouse, keyboard, touch panel, microphone, or the like. When the input interface is a touch panel, the input interface may be formed integrally with a display device such as a terminal device, a personal computer (PC), or a display connected to the medical information processing device 100. The input interface may be implemented by a terminal device, a personal computer (PC), or a display device (for example, a tablet terminal) capable of performing wireless communication with the medical information processing device 100. In the present specification, the input interface is not limited to one including the above-described physical operation parts such as a mouse or a keyboard. For example, an electrical signal corresponding to an input operation is received from an external input device provided separately from a terminal device, a personal computer (PC), or a medical information processing device 100 and electrical signal processing circuitry configured to output the electrical signal to the terminal device, the personal computer (PC), or the medical information processing device 100 is included as an example of an input interface.

In this way, the mapping processing function 142 is performed to extract medical terms from the text data and generate a mapped ontology in which the extracted medical terms are mapped to the corresponding entities constituting the basic medical ontology. The mapping processing function 142 is performed to output the generated mapped ontology to the identification function 144. Here, the mapped ontology generated in the mapping processing function 142 and output to the identification function 144 includes an entity mapped to the extracted medical term and only the higher-level entity connected to the entity mapped to the medical term (may include the information entity). That is, the mapped ontology is a medical ontology having a configuration in which entities not related to the medical term are omitted from the basic medical ontology configuration shown in FIG. 2. Thereby, the identification function 144 can be performed to identify medical information (medical words and medical terms) that are highly important for the disease that the diagnosis target patient is suffering from on the basis of a mapped ontology composed only of entities related to the medical term extracted from the text data. A mapped ontology is an example of a “modified medical ontology.”

In the above-described mapping process, a case where the mapping processing function 142 is performed to map the extracted medical term to a corresponding entity in the basic medical ontology has been described. However, the mapping process in which the mapping processing function 142 is performed to map medical terms to the basic medical ontology is not limited to a process of simply mapping the extracted medical data to the corresponding entity. For example, the mapping processing function 142 may be performed to map the extracted medical term to the basic medical ontology using a clustering method that is an unsupervised learning method or a supervised learning method. Here, another example of a mapping process of mapping a medical term to a basic medical ontology using a learning method will be described in the mapping processing function 142.

### First Modified Example of Mapping Process

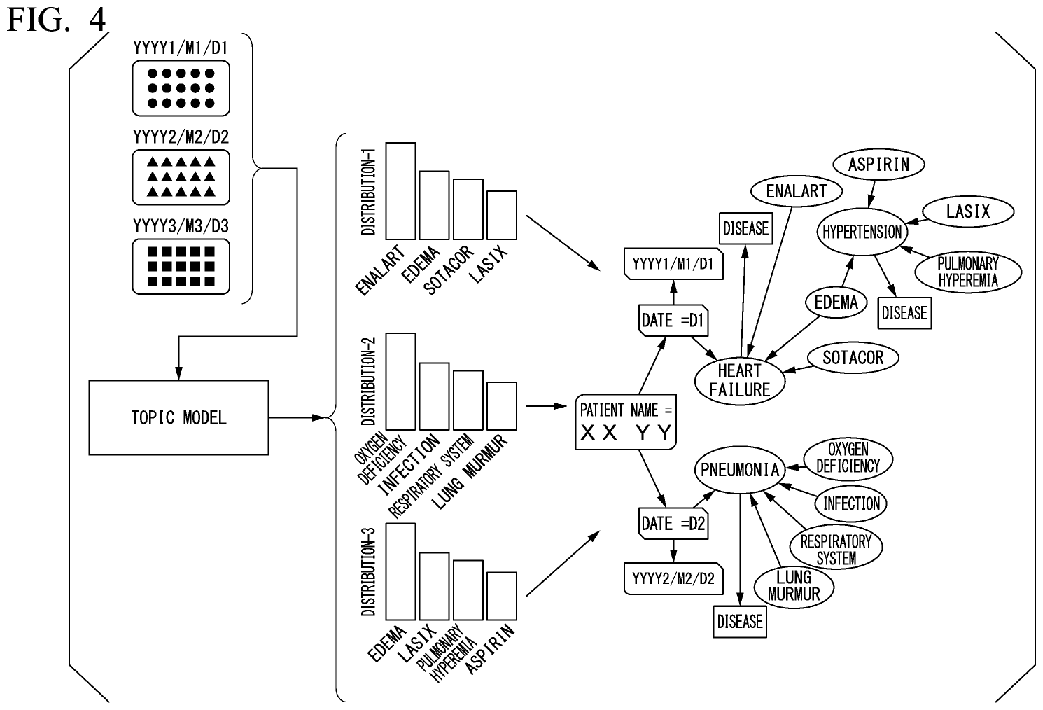

FIG. 4 is a diagram schematically showing another example (first modified example) of a mapping process of the mapping processing function 142 provided in the medical information processing device 100 according to the embodiment. In the mapping process of the first modified example, first, the mapping processing function 142 is performed to obtain a probability distribution in which each medical term appears in the text data output in the medical data acquisition function 120, for example, according to latent Dirichlet allocation (LDA) that implements a topic model. Also, the mapping processing function 142 is performed to map a plurality of medical terms appearing with a high probability (for example, a prescribed number of medical terms from the highest probability and the like) to a basic medical ontology according to a clustering method that is an unsupervised learning method. In FIG. 4, for example, an example of a flow of a process of a case where a probability distribution in which a medical term included in the text data of the diagnosis target patient (here, text data for different 3 days) recorded on different dates appears is obtained by the topic model (here, distributions 1 to 3) and a mapped ontology is generated by mapping four medical terms from the medical term with the highest probability to the basic medical ontology in each distribution is schematically shown.

The mapping processing function 142 is performed to output a mapped ontology (may have time information in the mapped ontology) generated in the mapping process of the first modified example to the identification function 144.

### Second Modified Example of Mapping Process

In the mapping process of the second modified example, the mapping processing function 142 is performed to map the extracted medical terms to the basic medical ontology using, for example, a machine learning function based on artificial intelligence (AI). At this time, the mapping of medical terms in the mapping processing function 142 is performed using a trained model trained by, for example, a graph convolutional neural network for a relationship (a graph structure) of medical information indicated in the basic medical ontology. The trained model is, for example, a trained model trained to output an entity mapped to the input medical term using a graph convolutional network (GCN) in which machine learning technology of a convolutional neural network (CNN), a deep neural network (DNN), or the like is applied to graph data. The CNN is a neural network in which several layers such as a convolution layer and a pooling layer are connected. The DNN is a neural network in which layers of any form are connected in multiple layers. A trained model is generated in machine learning using, for example, a machine learning model using a calculation device (not shown). In the calculation device (not shown), when the trained model is generated, text data included in medical data (electronic medical records) of another patient previously diagnosed or a diagnosis target patient and a basic medical ontology (information indicating the relationship of medical information indicated in the basic medical ontology) is input as input data and, for example, the mapped ontology corresponding to the other patient previously diagnosed or the diagnosis target patient and the like are input as training data to the output side of the trained model. On the input side when the calculation device (not shown) generates the trained model, for example, information indicating the specificity of the medical term to be described below, information indicating the co-occurrence degree of the medical term, or the like may be input as input data. On the output side when the calculation device (not shown) generates a trained model, for example, information indicating a representative entity representing the mapped ontology to be described below, such as the name of a disease assumed from the medical term, may be input as training data.

The mapping processing function 142 is performed to output the mapped ontology generated in the mapping process of the second modified example (may have time information in the mapped ontology) to the identification function 144.

The mapping processing function 142 is an example of a “mapping processor.”

The identification function 144 is performed to identify medical information satisfying a specific condition for the diagnosis target patient, such as the name of the disease suffered by the diagnosis target patient and information of high importance, on the basis of the mapped ontology output by the mapping processing function 142. More specifically, the identification function 144 is performed to trace the adjacency of the entity to which the medical term has been mapped in the mapping processing function 142 in the mapped ontology and form a cluster in which a label indicating a prescribed connection frequency based on the traced adjacency is connected to a target entity. At this time, the number of entities to which the identification function 144 is performed to trace adjacencies in the mapped ontology may be set in advance, for example, up to n (where n is a natural number) which is the number of adjacent entities or the like. In other words, the hierarchy of entities to which the identification function 144 is performed to trace adjacencies in the mapped ontology may be set in advance, such as, for example, up to entities below n layers. That is, restrictions on a method of tracing adjacencies in the mapped ontology in the identification function 144 may be provided. Also, the identification function 144 is performed to identify an entity indicating medical information indicating a specific condition in a formed cluster as an entity representing the formed cluster, i.e., an entity representing a mapped ontology (hereinafter referred to as a representative entity), using a clustering method, which is an unsupervised learning method. For example, the identification function 144 is performed to identify, for example, a higher-level entity having a large number of connected lower-level entities (lower-level entities to which medical terms are mapped) as a representative entity. A representative entity is an example of “specific information.”

The identification function 144 may be performed to identify the higher-level entity to which the specific category entity is connected as a representative entity. For example, when there are a plurality of higher-level entities connected to the category entity of “disease” and a plurality of higher-level entities connected to the category entity of “drug” within the mapped ontology, the identification function 144, for example, may be performed to identify a representative entity from higher-level entities connected to the same category entity (common category entity) such as the category entity of “disease.” At this time, the identification function 144 is performed to perform a switching process of determining which category entity is connected to a representative entity identified from among the higher-level entities, for example, in accordance with a type of medical word, i.e., a category, designated when the primary care physician operates the input interface (not shown) provided in the medical information processing device 100.

As described above, the process of identifying a representative entity in the identification function 144 is not limited to the process of identifying a higher-level entity to which a large number of lower-level entities are connected (to which medical terms are mapped) during a process of tracing the adjacency of entities on the mapped ontology. For example, the identification function 144 may be performed to identify a representative entity on the basis of the number of times each medical term appears (the number of appearances) in the text data output by the medical data acquisition function 120. That is, when the same medical term appears a plurality of times in the text data, the identification function 144 may be performed to identify the medical term having a large number of appearances as a medical term having high importance and identify the higher-level entity connected to the lower-level entity to which the medical term is mapped as a representative entity.

Here, some examples of a process of identifying a representative entity in the identification function 144 (an identification process) will be described. In the following description, it is assumed that the higher-level entity of the name of the disease having high importance is identified as a representative entity. In the following description, a lower-level entity to which a medical term is mapped is referred to as a “medical term” to distinguish it from a lower-level entity to which a medical term is not mapped. The higher-level entity of the name of the disease of high importance is an example of “first medical information.” The lower-level entity to which the medical term is mapped is an example of “second medical information.”

### [First Identification Process]

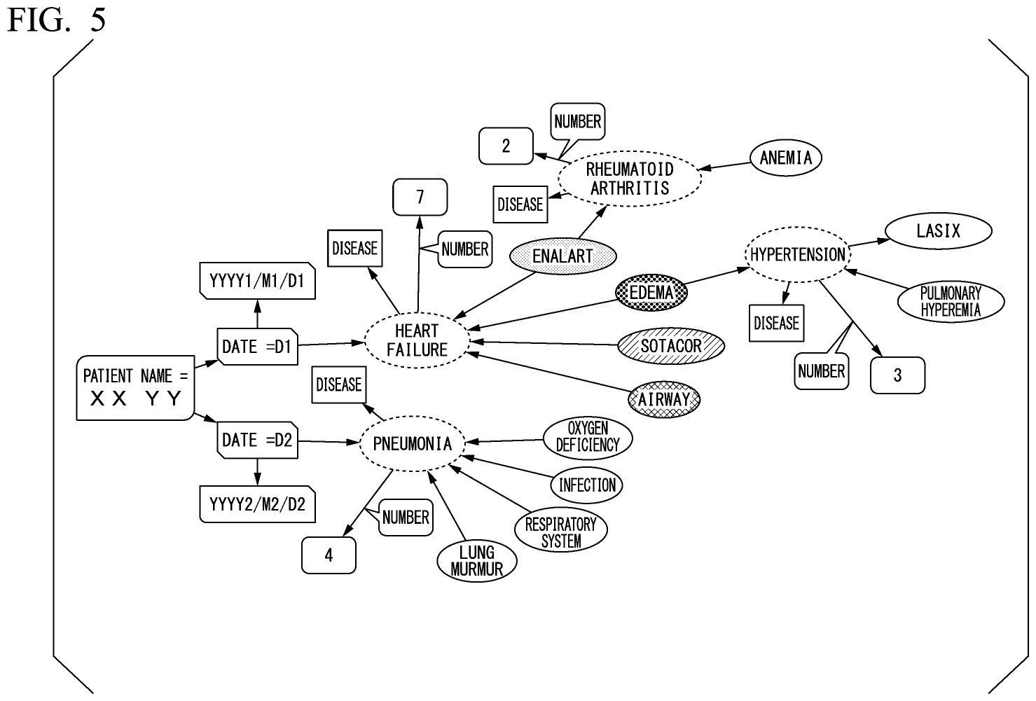

FIG. 5 is a diagram schematically showing an example of a first identification process of the identification function 144 provided in the medical information processing device 100 according to the embodiment. In FIG. 5, a state in which the identification function 144 is performed to perform the first identification process is schematically shown with respect to an example of a mapped ontology generated in the mapping processing function 142.

In the first identification process, in the mapped ontology, a higher-level entity of the name of the disease having a large number of connected medical terms and a large number of connected lower-level entities is designated as a higher-level entity having higher importance and identified as a representative entity. For this reason, the identification function 144 is performed to trace the adjacency of each medical term in the mapped ontology and counts the number of connected medical terms and the number of connected lower-level entities with respect to each higher-level entity. Also, the identification function 144 is performed to form a cluster in which labels indicating a counted medical term count value and a counted lower-level entity count value as a connection frequency are connected to each higher-level entity. At this time, the identification function 144 is connected to each higher-level entity using a label attribute as “number.”

In FIG. 5, in the mapped ontology, an example of a case where the label of a count value=“7” is connected to the higher-level entity where the disease name is “heart failure,” the label of a count value=“4” is connected to the higher-level entity whose disease name is “pneumonia,” the label of a count value=“3” is connected to the higher-level entity whose disease name is “hypertension,” and the label of a count value=“2” is connected to the higher-level entity whose disease name is “rheumatoid arthritis” is shown. Here, the count value=“7” of the label connected to the higher-level entity of “heart failure” is a count value obtained by counting the number of medical terms of each of “edema” whose relationship is “symptom,” “airway” whose relationship is “site,” and “Enalart” and “Sotacor” whose relationship is “therapeutic drug,” and “digoxin,” “hydrochlorothiazide,” and “digoxin elixir” whose relationship is “therapeutic drug” omitted in FIG. 5 connected to the higher-level entity. Likewise, the count value=“4” of the label connected to the higher-level entity of “pneumonia” is a count value obtained by counting the number of medical terms “lung murmur” and “oxygen deficiency” whose relationship is “symptom,” “infection” whose relationship is “cause,” and “respiratory system” whose relationship is “therapeutic method” connected to the higher-level entity. Likewise, the count value=“3” of the label connected to the higher-level entity of “hypertension” is a count value obtained by performing a count process for each of the medical term “edema” whose relationship is “symptom,” the lower-level entity of “pulmonary hypertension,” and the lower-level entity of “Lasix” whose relationship is “therapeutic drug” connected to the higher-level entity. Likewise, the count value=“2” of the label connected to the higher-level entity of “rheumatoid arthritis” is a count value obtained by performing a count process for each of the lower-level entity of “anemia” whose relationship is “symptom” and the medical term “Enalart” whose relationship is “therapeutic drug” connected to the higher-level entity. Although the number of category entities connected to each higher-level entity is not counted in the example shown in FIG. 5, the identification function 144 may be performed to count the number of category entities and reflect the counted number of category entities in the count value of each label.

Also, the identification function 144 is performed to designate the higher-level entity to which the label having the largest count value is connected as the higher-level entity having the highest importance and identify the designated higher-level entity as a representative entity. In the example shown in FIG. 5, the higher-level entity of “heart failure” to which the label of the largest count value=“7” is connected is identified as a representative entity.

The identification function 144 may be performed to identify the entity as a representative entity for each recording date on which text data is recorded (described) on the basis of a “date” information entity indicating information of a time concept connected to the mapped ontology. For example, in the example shown in FIG. 5, the higher-level entity of “heart failure” to which the label of the largest count value=“7” in the relationship indicated in the mapped ontology of the series to which the information entity of “date=D1” is connected is identified as a representative entity corresponding to “date=D1” and the higher-level entity of “pneumonia” to which the label of the largest count value=“4” in the relationship indicated in the mapped ontology of the series to which the information entity of “date=D2” is connected is identified as a representative entity corresponding to “date=D2.”

### [Second Identification Process]

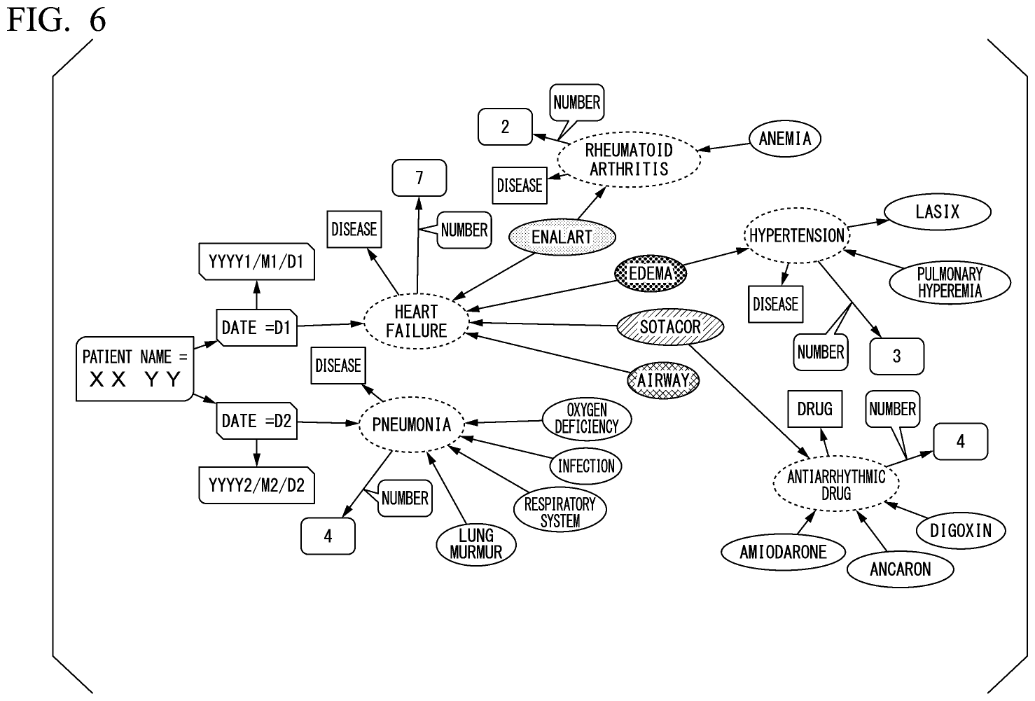

FIG. 6 is a diagram schematically showing an example of a second identification process of the identification function 144 provided in the medical information processing device 100 according to the embodiment. In FIG. 6, a state in which the identification function 144 is performed to perform the second identification process with respect to an example of a mapped ontology generated in the mapping processing function 142 is shown.

In the second identification process, in the mapped ontology, for example, a higher-level entity having a large number of connected medical terms and a large number of connected lower-level entities among the higher-level entities to which a specific category entity designated by the primary care physician is connected is designated as a higher-level entity having higher importance and identified as a representative entity. For this reason, even in the second identification process, as in the first identification process, the identification function 144 is performed to count the number of connected medical terms and the number of connected lower-level entities connected to each higher-level entity and form a cluster in which a label indicated using a medical term count value and a lower-level entity count value as the connection frequency is connected to each higher-level entity.

In FIG. 6, as in the first identification process, an example of a case where the labels of the medical term count value and the lower-level entity count value are connected to each higher-level entity in the mapped ontology is shown. The mapped ontology shown in FIG. 6 includes a higher-level entity whose therapeutic drug type (generic term) is “antiarrhythmic drug” with respect to the mapped ontology shown in FIG. 5. For this reason, the identification function 144 is also performed to connect the labels indicating the medical term count value and the lower-level entity count value to the higher-level entity of “antiarrhythmic drug” in addition to the labels of the medical term count value and lower-level entity count value connected to each higher-level entity of the disease name in the first identification process. In FIG. 6, an example of a case where the label of a count value=“4” is connected to the higher-level entity of “antiarrhythmic drug” is shown. Here, the count value=“4” of the label connected to the higher-level entity of “antiarrhythmic drug” is a count value obtained by performing a count process for each of the medical term “Sotacor” whose relationship is “therapeutic drug” and the lower-level entities of “amiodarone,” “Ancaron,” and “digoxin” connected to the higher-level entity. In the example shown in FIG. 6, when the number of category entities connected to each higher-level entity is counted and the counted number is reflected in the count value of each label, the identification function 144 causes a count value obtained by counting the number of category entities of “drug” to be reflected in the count value of the label connected to the higher-level entity of “antiarrhythmic drug.”

Also, the identification function 144, for example, is performed to designate the higher-level entity to which the label of the largest count value is connected among the higher-level entities to which the type of medical word designated by the primary care physician, i.e., the same category entity (common category entity), is connected as a higher-level entity having highest importance and identify the designated higher-level entity as a representative entity. For example, when a category entity of “disease” is designated in the example shown in FIG. 6, the identification function 144 is performed to identify the higher-level entity of “heart failure” to which the label of the largest count value=“7” is connected among higher-level entities to which the category entity of “disease” is connected as a representative entity as in the first identification process. At this time, even if the count value of the label connected to the higher-level entity of “antiarrhythmic drug” is greater than the count value of the label connected to the higher-level entity of “heart failure,” the identification function 144 is performed to identify the higher-level entity of “heart failure” as a representative entity without identifying the higher-level entity of “antiarrhythmic drug” to which the category entity of “drug” is connected as a representative entity because the designated category entity is “disease.”

On the other hand, when a category entity of “drug” is designated, the identification function 144 is performed to identify the higher-level entity of “antiarrhythmic drug” to which the label of the largest count value=“4” among the higher-level entities to which the category entity of “drug” is connected as a representative entity. However, when the entity is identified as a representative entity for each record date in which text data is recorded (described) on the basis of the “date” information entity indicating information about a time concept connected to the mapped ontology, there is no higher-level entity to which the category entity of “drug” is connected in the series connected to “date=D2.” For this reason, the identification function 144 is performed so that the higher-level entity to which the category entity of “drug” is connected is not identified (cannot be identified) as a representative entity. In this case, the identification function 144 may be performed to identify the higher-level entity of “pneumonia” to which the label of the largest count value=“4” in the series connected to “date=D2” is connected as a representative entity corresponding to “date=D2” instead thereof.

### [Third Identification Process]

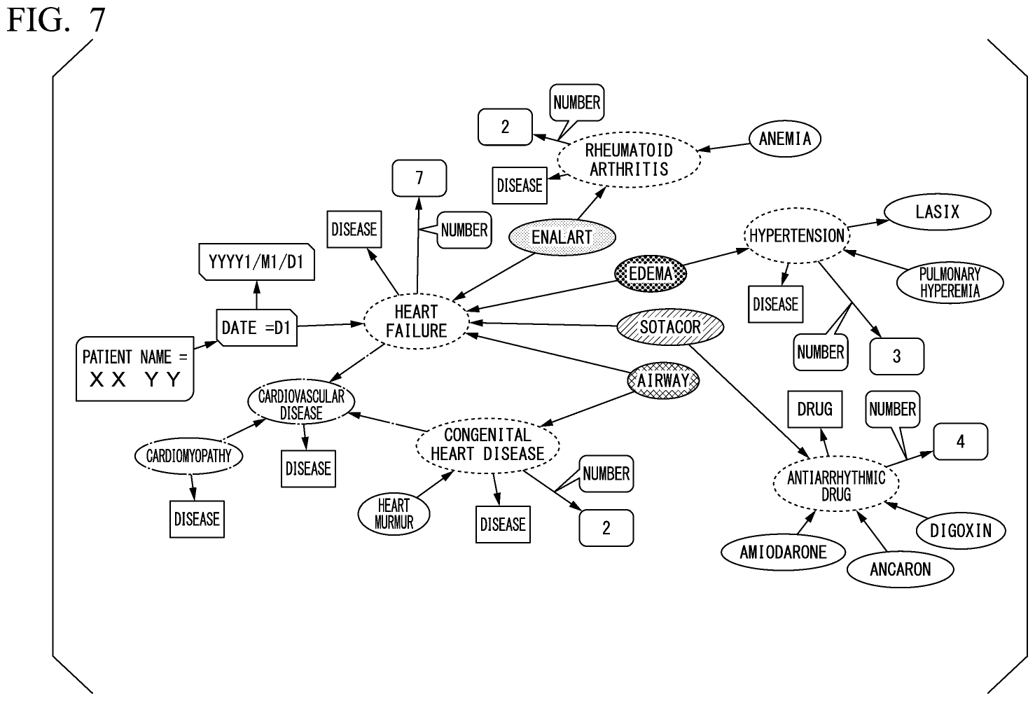

FIG. 7 is a diagram schematically showing an example of a third identification process of the identification function 144 provided in the medical information processing device 100 according to the embodiment. In FIG. 7, a state in which the identification function 144 is performed to perform the third identification process with respect to an example of a mapped ontology generated in the mapping processing function 142 is schematically shown. In an example of the mapped ontology shown in FIG. 7, the entity of the series connected to “date=D2” is omitted.

In the third identification process, in the mapped ontology, the entity of the higher-level concept in each higher-level entity to which the medical term and the lower-level entity are connected, i.e., the entity higher than the higher-level entity (hereinafter referred to as a “higher-level concept entity”), is traced and the higher-level entity having a larger number of connected medical terms and a larger number of connected lower-level entities among higher-level entities connected to the same higher-level concept entity (the common higher-level concept entity) is designated as a higher-level entity having higher importance and identified as a representative entity. For this reason, even in the third identification process, as in the first identification process and the second identification process, the identification function 144 is performed to count the number of connected medical terms and the number of connected lower-level entities connected to each higher-level entity and form a cluster in which a label indicated using a medical term count value and a lower-level entity count value as the connection frequency is connected to each higher-level entity. A higher-level concept entity higher than a higher-level entity is an example of “first medical information with a common higher-level concept.”

In FIG. 7, an example of a case where the labels of the medical term count value and the lower-level entity count value are connected to each higher-level entity in the mapped ontology is shown as in the first identification process or the second identification process. In the mapped ontology shown in FIG. 7, the higher-level entity with the disease name “congenital heart disease,” the higher-level entity of “cardiomyopathy,” and the higher-level entity of “cardiovascular disease” are included with respect to each entity in the series connected to “date=D1” shown in FIG. 6. The identification function 144 is performed to connect a label of a count value=“2” to the higher-level entity of “congenital heart disease.” Here, the count value=“2” of the label connected to the higher-level entity of “congenital heart disease” is a count value obtained by performing a count process for each of the medical term “airway” whose relationship is “site” and the lower-level entity of “heart murmur” whose relationship is “symptom” connected to the higher-level entity. In the example shown in FIG. 7, when the number of category entities connected to each higher-level entity is counted and the counted number is reflected in the count value of each label, the identification function 144 causes a count value obtained by counting the number of category entities of “disease” to be reflected in the count value of the label connected to the higher-level entity of “congenital heart disease.”

Also, the identification function 144 is performed to trace a higher-level concept entity of each higher-level entity and extract a higher-level entity connected to a common higher-level concept entity. In the example shown in FIG. 7, the higher-level entity of “congenital heart disease” and the higher-level entity of “cardiomyopathy” are entities having the same layer as the higher-level entity of “heart failure” and the higher-level entity of “cardiovascular disease” is one layer higher than each of the higher-level entities of “heart failure,” “congenital heart disease,” and “cardiomyopathy” connected thereto. That is, the higher-level entity of “cardiovascular disease” is a higher-level concept entity (common higher-level concept entity) for each of the higher-level entities of “heart failure,” “congenital heart disease,” and “cardiomyopathy.” For this reason, the identification function 144 is performed to extract a higher-level entity to which the label of the count value is connected from among the higher-level entities connected to the higher-level concept entity of “cardiovascular disease.” In the example shown in FIG. 7, the identification function 144 is performed to extract a higher-level entity of “heart failure” and a higher-level entity of “congenital heart disease.” The identification function 144 is performed to designate the higher-level entity to which the label of the largest count value is connected among the extracted higher-level entities as a higher-level entity having highest importance as in the first identification process or the second identification process and identify the designated higher-level entity as a representative entity. In the example shown in FIG. 7, the higher-level entity of “heart failure” to which the label of the largest count value=“7” is connected between the higher-level entity of “heart failure” and the higher-level entity of “congenital heart disease is identified as a representative entity.

### [Fourth Identification Process]

In the first to third identification processes, the identification function 144 is performed to identify a higher-level entity (a representative entity) having higher importance on the basis of connection frequencies (count values) for the medical term and the lower-level entity in the higher-level entity. On the other hand, in the fourth identification process, the importance of each higher-level entity is determined in consideration of the specificity of the medical term connected to each higher-level entity, i.e., on the basis of whether or not the medical term is a specific medical word, and the higher-level entity having higher importance is identified as the representative entity. In other words, in the fourth identification process, the importance of each higher-level entity is determined on the basis of the connection frequency including a value (feature value) indicating a feature of the connected medical term. A feature value of the medical term is, for example, a weight value (a weight factor) based on the number of higher-level entities to which this medical term can be connected. More specifically, the weight value of the medical term is designated as “1” when the number of higher-level entities that can be connected is one and decreases as the number of higher-level entities that can be connected increases. For example, when the category (the category entity) of medical words in a medical term is “drug” and the drug (the therapeutic drug) of this medical term corresponds to a specific disease or symptom (i.e., corresponds to a single disease or symptom), the weight value is designated as “1” because there is only one higher-level entity of “disease” or “symptom” to which this medical term can be connected (having a relationship as “therapeutic drug”). On the other hand, when the drug (the therapeutic drug) of this medical term corresponds to a plurality of diseases and symptoms, i.e., for example, it is a highly versatile therapeutic drug that is effective for many diseases and symptoms such as fever and pain, a reciprocal of the number of higher-level entities of all “diseases” and “symptoms” to which this medical term can be connected (similarly having a relationship as “therapeutic drug”) is designated as the weight value. This weight value is connected to the target entity in a basic medical ontology configuration, for example, using the label attribute as “weight.” The weight value is an example of a “feature term.”

In the fourth identification process, when the number of connected medical terms and the number of connected lower-level entities for each higher-level entity are counted, the identification function 144 is performed to designate a value multiplied by the weight value (a weighted value) as a count value. Thereby, in the fourth identification process, the count values obtained by counting the number of connected medical terms and the number of connected lower-level entities for each higher-level entity in the identification function 144 are determined in consideration of the specificity of the medical term with respect to the count values in the first to third identification processes. Also, in the fourth identification process, a cluster in which labels indicating the medical term count value and the lower-level entity count value counted by multiplying the weight value as the connection frequency are connected to each higher-level entity is formed. At this time, the identification function 144 is performed to connect the label attribute to each higher-level entity as a “weighted frequency.”

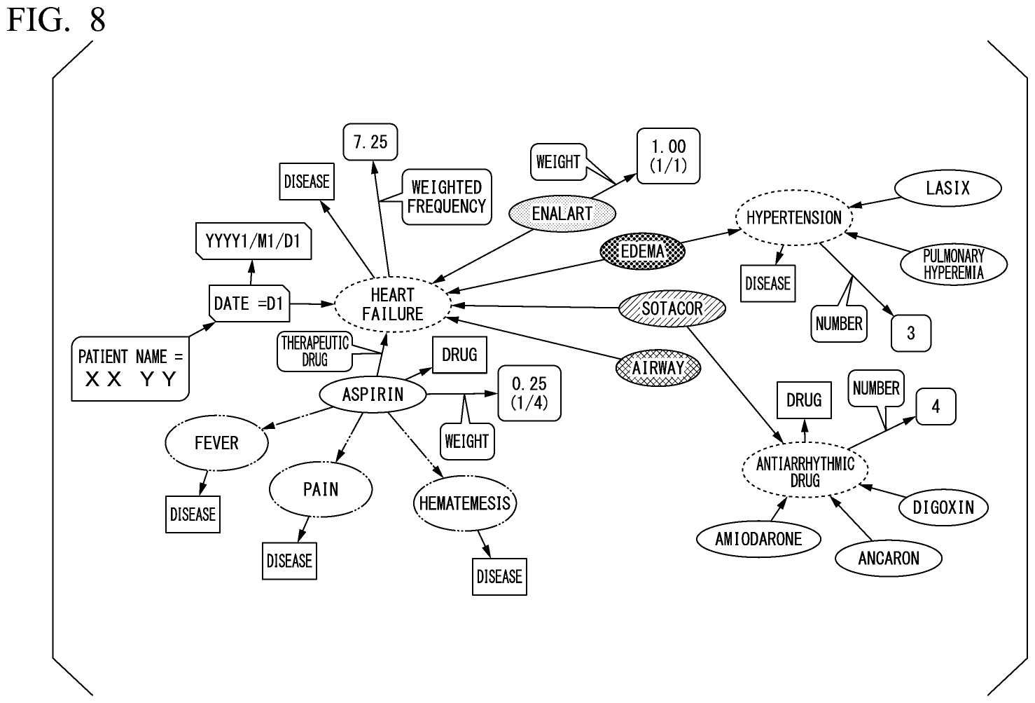

FIG. 8 is a diagram schematically showing an example of the fourth identification process of the identification function 144 provided in the medical information processing device 100 according to the embodiment. In FIG. 8, a state in which the identification function 144 is performed to perform the fourth identification process with respect to an example of a mapped ontology generated in the mapping processing function 142 is schematically shown. In an example of the mapped ontology shown in FIG. 8, a category entity with a medical word category of “disease” is connected to a higher-level entity with a disease name of “heart failure,” a medical term “edema” whose relationship is “symptom” is connected, a medical term “airway” whose relationship is “site” is connected, and medical terms “Enalart,” “Sotacor,” and “Aspirin” whose relationship is “therapeutic drug” and medical terms “digoxin,” “hydrochlorothiazide,” and “digoxin elixir” whose relationship is “therapeutic drug” omitted in FIG. 8 are connected. Furthermore, in an example of the mapped ontology shown in FIG. 8, the medical term “edema” is connected to the higher-level entity of the disease name of “hypertension,” a category entity of the medical word category of “disease” is connected in the higher-level entity of “hypertension,” the lower-level entity of “pulmonary hypertension” whose relationship is “symptom” is connected, and the lower-level entity of “Lasix” whose relationship is “therapeutic drug” is connected. Furthermore, in an example of the mapped ontology shown in FIG. 8, the medical term “Sotacor” is connected to the higher-level entity of the type of therapeutic drug (generic term) of “antiarrhythmic drug,” a category entity of the medical word category of “drug” is connected in the higher-level entity of “antiarrhythmic drug,” and the lower-level entities of “amiodarone,” “Ancaron,” and “digoxin” indicating the name of a therapeutic drug having efficacy similar to that of an antiarrhythmic drug are connected. Furthermore, in an example of the mapped ontology shown in FIG. 8, the higher-level entities of “fever,” “pain,” and “hematemesis” indicating “disease” and “symptom” similarly corresponding to the medical term “aspirin” are connected.

In an example of the mapped ontology shown in FIG. 8, an example of a case where the label of a weight value=“0.25” is connected to “aspirin” having a relationship of “therapeutic drug” to the higher-level entity of the disease name of “heart failure” and the label of a weight value=“1.00” is connected to “Enalart” is shown. In an example of the mapped ontology shown in FIG. 8, for ease of description, the label of the weight value=“1.00” is connected to “Sotacor” having a relationship of “therapeutic drug” for the higher-level entity of the disease name of “heart failure.” Here, the weight value=“0.25” of the label connected to the medical term “aspirin” indicates that three higher-level entities of “fever,” “pain,” and “hematemesis” are connected as the higher-level entity of “disease” or “symptom” corresponding to this medical term in addition to “heart failure,” i.e., four higher-level entities are connected. On the other hand, the weight value=“1.00” of the label connected to the medical term “Sotacor” indicates that only “heart failure,” i.e., only one higher-level entity, is connected as the higher-level entity of “disease” or “symptom” corresponding to this medical term.

In the fourth identification process, the identification function 144 is performed to count the number of connected medical terms and the number of connected lower-level entities multiplied by the weight value for each higher-level entity and connect the counted numbers as a label indicating a connection frequency (a weighted frequency). In FIG. 8, a label of a count value=“7.25” is connected to the higher-level entity whose disease name is “heart failure” in the mapped ontology. Here, the count value=“7.25” of the label connected to the higher-level entity of “heart failure” is a count value obtained by counting the value of “aspirin” as a weight value=“0.25” when the number of medical terms “edema,” “airway.” “Enalart,” “Sotacor,” and “aspirin” and the number of medical terms “digoxin,” “hydrochlorothiazide,” and “digoxin elixir” omitted in FIG. 8 connected to the higher-level entity are counted. In FIG. 8, it is assumed that the labels of simple count values of the number of connected medical terms and the number of connected lower-level entities are connected to higher-level entities of “hypertension” and “antiarrhythmic drug.”

Also, the identification function 144 is performed to designate the higher-level entity to which the label having the largest count is connected as the higher-level entity having the highest importance and identifies the designated higher-level entity as a representative entity. In the example shown in FIG. 8, the higher-level entity of “heart failure” to which the label of the largest count value=“7.25” is connected is identified as a representative entity.

### [Fifth Identification Process]

In the fourth identification process, the identification function 144 is performed to identify a higher-level entity (a representative entity) having higher importance on the basis of count values (weighted frequencies) of the number of medical terms and the number of lower-level entities in the higher-level entity counted by assigning a weight in consideration of the specificity of the medical term connected to each higher-level entity. On the other hand, in the fifth identification process, a higher-level entity having higher importance is identified as a representative entity by obtaining a co-occurrence degree indicating the proportion of medical terms that are simultaneously extracted from the text data included in the medical data (electronic medical records) and determining the importance of each higher-level entity in consideration of the obtained co-occurrence degree. The co-occurrence degree can be obtained by, for example, a Jaccard coefficient or the like. More specifically, the co-occurrence degree is, for example, obtained by dividing the number of items of text data including both a certain medical term A and a certain medical term B by the number of items of text data including one or both of the medical term A and the medical term B. The co-occurrence degree may be obtained when the medical term extracted in the mapping processing function 142 is mapped to the medical ontology or the identification function 144 may be performed to obtain the co-occurrence degree with reference to text data when the adjacency of the entity to which the medical term is mapped in the mapped ontology is traced. The co-occurrence degree may be obtained, for example, in a processing function of obtaining the co-occurrence degree of the medical term included in the text data provided in the medical data processing function 140.

In the fifth identification process, the identification function 144 is performed to designate a value including the co-occurrence degree between the medical terms as a count value when the number of connected medical terms and the number of connected lower-level entities for each higher-level entity are counted. Thereby, count values obtained by counting the number of connected medical terms and the number of connected lower-level entities for each higher-level entity in the identification function 144 in the fifth identification process are determined in consideration of the co-occurrence degree between the medical terms with respect to the count values in the first to third identification processes. In other words, the count value in the fifth identification process is obtained by adding a weight representing the strength of the connection between medical terms on the basis of the co-occurrence degree with respect to the count values in the first to third identification processes. Also, in the fifth identification process, a cluster in which labels indicating the count values of the number of medical terms and the number of lower-level entities counted together with the co-occurrence degree as the connection frequencies are connected to each higher-level entity is formed. At this time, the identification function 144 makes a connection to each higher-level entity using the label attribute as a “co-occurrence frequency.”

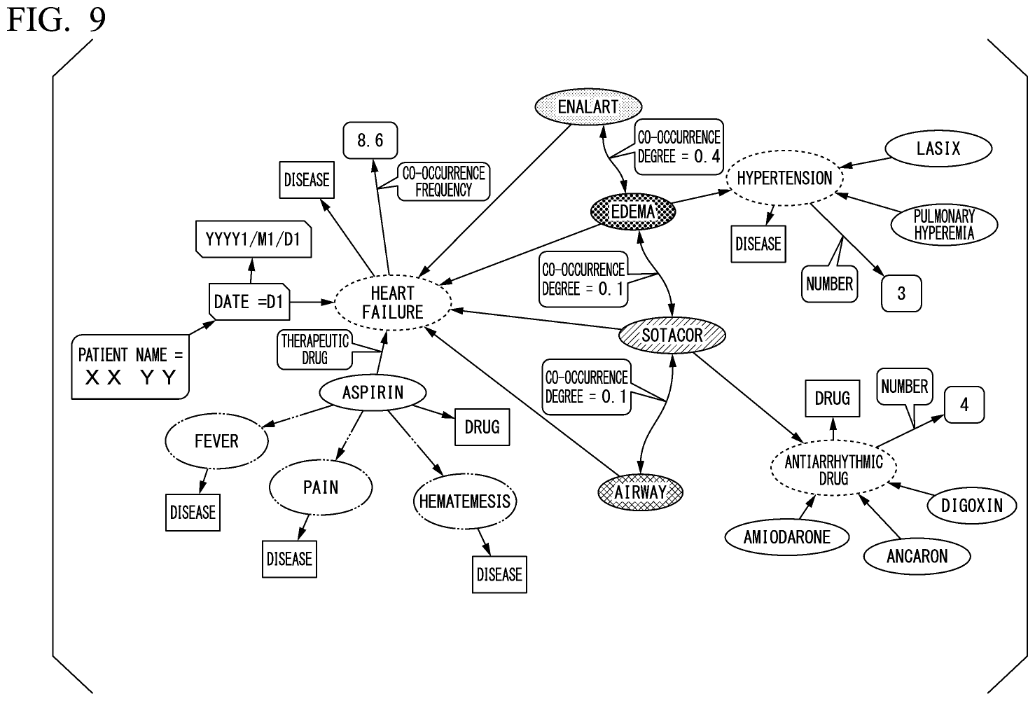

FIG. 9 is a diagram schematically showing an example of the fifth identification process of the identification function 144 provided in the medical information processing device 100 according to the embodiment. In FIG. 9, as in the fourth identification process, an example of a case where a medical term “aspirin” having a relationship of “therapeutic drug” with respect to the higher-level entity whose disease name is “heart failure” is connected in the mapped ontology is shown. However, in the mapped ontology shown in FIG. 9, for ease of description, an example of a case where the labels of the weight values are not connected, i.e., the weight value in each medical term is “1.00” is shown. Also, in the mapped ontology shown in FIG. 9, an example of a case where the co-occurrence degree between the medical term “Enalart” and the medical term “edema” is “0.4,” the co-occurrence degree between the medical term “edema” and the medical term “Sotacor” is “0.1,” and the co-occurrence degree between the medical term “Sotacor” and the medical term “airway” is “0.1” is shown.

In the fifth identification process, the identification function 144 is performed to perform a count process together with the co-occurrence degree and make a connection using a count value as a label indicating the connection frequency (the co-occurrence frequency) when the number of connected medical terms and the number of connected lower-level entities for each higher-level entity are counted. In FIG. 9, in the mapped ontology, a label of a count value=“8.6” is connected to the higher-level entity whose disease name is “heart failure.” Here, the count value=“8.6” of the label connected to the higher-level entity of “heart failure” is a count value obtained by adding a co-occurrence degree between the medical terms “Enalart,” “edema,” “Sotacor,” and “airway” to the count value of each of the medical terms “edema,” “airway,” “Enalart,” “Sotacor,” and “aspirin” and the medical terms “digoxin,” “hydrochlorothiazide,” and “digoxin elixir” omitted in FIG. 9 connected to the higher-level entity. In FIG. 9, for the higher-level entities of “hypertension” and “antiarrhythmic drug,” the labels of the connected medical term count value and the connected lower-level entity count value are connected. This is because neither of the higher-level entities of “hypertension” and “antiarrhythmic drug” is simultaneously connected to two medical terms that are co-related.

Also, the identification function 144 is performed to designate the higher-level entity to which the label having the largest count value is connected as the higher-level entity having the highest importance and identify the designated higher-level entity as a representative entity. In the example shown in FIG. 9, the higher-level entity of “heart failure” to which the label of the largest count value=“8.6” is connected is identified as a representative entity.

In this way, the identification function 144 is performed to identify a representative entity representing the mapped ontology, such as information having high importance, on the basis of the mapped ontology. The identification function 144 is performed to output information indicating the identified representative entity and the formed cluster to the information provision function 160. At this time, the identification function 144, for example, is performed to output information including the information indicating the representative entity and the formed cluster such as a mapped ontology in which the label indicating the identified representative entity is further connected to the mapped ontology in which the label indicating the connection frequency is connected to each higher-level entity to the information provision function 160. The identification function 144 may be performed to output information such as the name of the disease in the identified representative entity to the information provision function 160 separately from the formed cluster.

The identification function 144 is an example of an “identifier.”

The information provision function 160 is performed to generate provision information for providing information about the disease suffered by the diagnosis target patient to the primary care physician on the basis of information indicating a representative entity output by the medical data processing function 140 (more specifically, a cluster formed in the identification function 144). The information provision function 160 is performed to provide information about the disease suffered by the diagnosis target patient to the primary care physician by generating a display image for displaying display content indicating information about the disease or diagnosis, for example, on the basis of the cluster formed in the identification function 144, and causing a display device (not shown) connected to the medical information processing device 100 to display the generated display image. The information provision function 160 is connected to the network NW, for example, by controlling a communicator (not shown), and may be performed to provide information about the disease suffered by the diagnosis target patient to the primary care physician by transmitting a display image generated for a terminal device or the like used when the primary care physician confirms information about the disease of the diagnosis target patient and causing the display device provided in the terminal device or connected to the terminal device to display the display image.

The information provision function 160 is an example of a “display controller.”

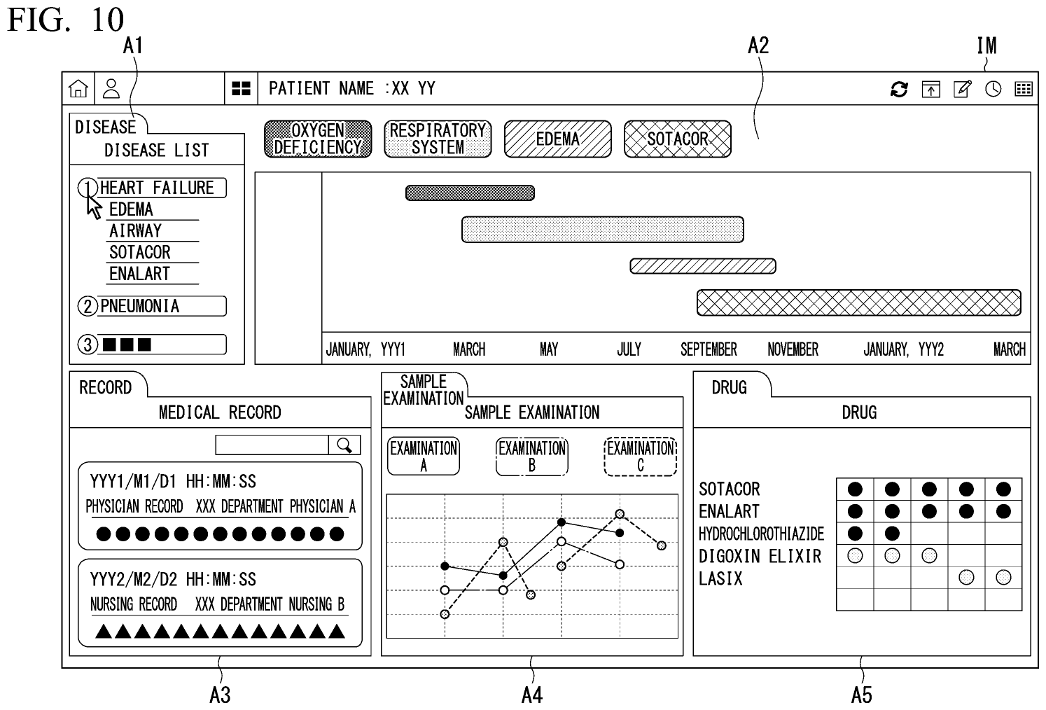

FIG. 10 is a diagram showing an example of a display screen on which the medical information processing device 100 according to the embodiment provides information. In FIG. 10, an example of a display screen IM in which display content indicating the information to be provided is displayed on the display device is shown. The display screen IM shown in FIG. 10 shows, for example, an example in which information based on a cluster formed by the identification function 144 is presented to each of five information presentation areas A. In the display screen IM, the name of the disease of the representative entity (the higher-level entity) identified in the identification function 144 is presented to the information presentation area A1 and the information of the lower-level entity connected to the representative entity in the cluster is shown. Although the names of diseases are listed and shown in the information presentation area A1 in the display screen IM shown in FIG. 10, the information shown in the information presentation area A1, for example, may be shown by listing therapeutic drugs prescribed to the diagnosis target patient or may be shown by listing therapeutic methods performed on the diagnosis target patient. In the display screen IM, information presentation areas A2 to A5 show time information and detailed information in the representative entity and the lower-level entity connected to the representative entity. In the information presentation areas A2 to A5, for example, time information about the lower-level entity connected to the higher-level entity (the representative entity) of the designated disease is shown when the primary care physician has designated the disease (herein, “heart failure”) displayed in the information presentation area A1 by operating the input interface (not shown) provided in the medical information processing device 100. In other words, in the information presentation areas A2 to A5, information filtered in accordance with an instruction from the primary care physician is shown. The information presentation areas A2 to A5 may be configured to present information extracted from the cluster or may be configured to present information extracted with reference to the original medical data (electronic medical records) forming the cluster, the basic medical ontology, or the like. The primary care physician is allowed to confirm information about the disease of the diagnosis target patient together with previous diagnosis results and the like through the display screen IM.

Although an example of a case where information is presented (displayed) to each of the five information presentation areas A including the information presentation areas A1 to A5 is shown in the display screen IM shown in FIG. 10, this is only an example and a method in which the medical information processing device 100 provides information to the primary care physician may be another method. For example, the information provision function 160 may cause an examination image of the diagnosis target patient to be presented (displayed) to the information presentation areas A2 to A5 shown in the display screen IM or another information presentation area A. That is, the method in which the information provision function 160 provides information to the primary care physician through the display screen may be any method as long as it is a method of providing information so that the primary care physician can easily confirm the information by filtering and presenting information about the disease that the diagnosis target patient is suffering from.

### Modified Example of Fifth Identification Process

Meanwhile, in the fifth identification process, the identification function 144 is performed to obtain a co-occurrence degree indicating the proportion of medical terms simultaneously extracted from the text data included in the medical data (electronic medical records) and identify a higher-level entity (a representative entity) having higher importance determined in consideration of the obtained co-occurrence degree. In other words, in the fifth identification process, a higher-level entity connected to many medical terms having a high co-occurrence degree is identified as a representative entity. Here, when a therapeutic process for a diagnosis target patient is performed, for example, a case where a new medical term is extracted from the text data of the current medical data without being extracted from the previous medical data (electronic medical records) or a medical term extracted from the text data of the medical data of the past time (for example, the past 1 year) and not extracted for some time is extracted again is conceivable. In the fifth identification process for such a medical term, a co-occurrence degree associated with other medical terms is not obtained, i.e., a co-occurrence degree=“0.0.” However, a case where newly extracted or re-extracted medical terms include, for example, a medical term indicating another disease occurring in the diagnosis target patient different from a disease currently being treated, a sudden change in the disease currently being treated, or a recurrence of a disease that has previously been suffered but treated is conceivable. Therefore, in a modified example of the fifth identification process, when a medical term has a co-occurrence degree=“0.0” but there is a newly (re)extracted medical term, the importance of each higher-level entity including this medical term is determined and the higher-level entity having higher importance is identified as a representative entity.

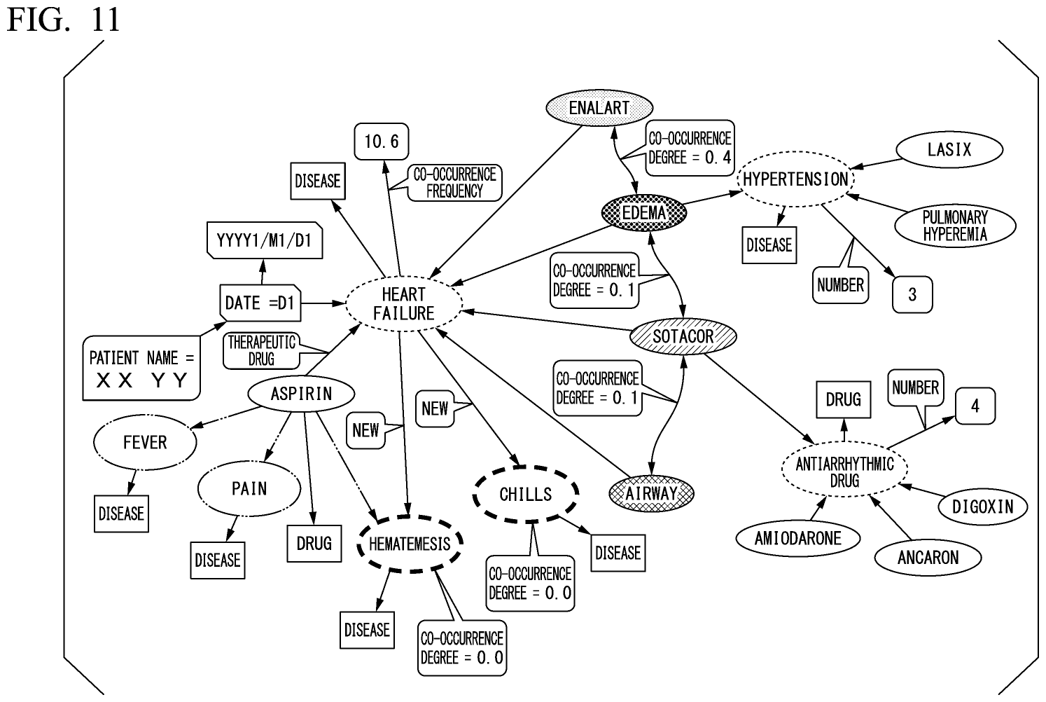

FIG. 11 is a diagram schematically showing an example of a modified example of the fifth identification process of the identification function 144 provided in the medical information processing device 100 according to the embodiment. In FIG. 11, as in the fifth identification process, an example in which the co-occurrence degree between the medical term “Enalart” and the medical term “edema” is “0.4,” the co-occurrence degree between the medical term “edema” and the medical term “Sotacor” are “0.1,” and the co-occurrence degree between the medical term “Sotacor” and the medical term “airway” are “0.1” is shown. Also, in the mapped ontology shown in FIG. 11, an example in which the medical term “hematemesis,” which is a higher-level entity of “disease” connected to the medical term “aspirin,” and the medical term “chills,” which is a higher-level entity of “disease,” are extracted newly (again) is shown. The medical terms “hematemesis” and “chills” are completely new medical terms that have not been extracted from the text data of medical data in the past time (for example, past 1 year) or medical terms that have not been extracted for some time (for example, the past 3 months) but have been extracted again from the text data of the current medical data. In this case, the medical term “hematemesis” and the medical term “chills” have a co-occurrence degree=“0.0.”

In the modified example of the fifth identification process, as in the fifth identification process, the identification function 144 is performed to perform a count process together with the co-occurrence degree and make a connection using a count value as a label indicating the connection frequency (the co-occurrence frequency) when the number of connected medical terms and the number of connected lower-level entities for each higher-level entity are counted. In FIG. 11, in the mapped ontology, a label of a count value=“10.6” is connected to the higher-level entity whose disease name is “heart failure.” Here, the count value=“10.6” of the label connected to the higher-level entity of “heart failure” is a count value obtained by adding a co-occurrence degree between the medical terms “Enalart,” “edema,” “Sotacor,” and “airway” to count values of the newly extracted medical terms “hematemesis” and “chills” in addition to the medical terms “edema,” “airway,” “Enalart,” “Sotacor,” and “aspirin” and the medical terms “digoxin,” “hydrochlorothiazide,” and “digoxin elixir” omitted in FIG. 11 connected to the higher-level entity. In FIG. 11, the higher-level entities of “hypertension” and “antiarrhythmic drug” are similar to those of the example of the fifth identification process shown in FIG. 9. For this reason, the identification function 144 is performed to identify “heart failure,” which is a higher-level entity to which the label of the largest count value (count value=“10.6”) is connected, as a representative entity even in the modified example of the fifth identification process.

In this way, the identification function 144 is performed to identify a representative entity representing a mapped ontology such as information having high importance on the basis of a mapped ontology including a medical term having a co-occurrence degree=“0.0” that is newly extracted (again) even in the modified example of the fifth identification process. In the modified example of the fifth identification process, the identification function 144 may be performed to designate a medical term that focuses on the medical terms that are newly extracted (again) (here, medical terms “hematemesis” and “chills”) as a medical term of interest and output the information of the medical term having high importance in the medical term of interest to the information provision function 160 together with information indicating the identified representative entity and the formed cluster. At this time, the identification function 144 is performed to determine, for example, the medical term “hematemesis” as a medical term having higher importance than the medical term “chills,” which is a higher-level entity of the same “disease,” and output the information of the medical term “hematemesis” as information of a medical term of interest having high importance to the information provision function 160. Thereby, the information provision function 160 is performed to generate a display image for providing the primary care physician with information about the disease that the diagnosis target patient is suffering from together with information about medical terms of interest having high importance and cause the display device (not shown) connected to the medical information processing device 100 to display the generated displayed image.

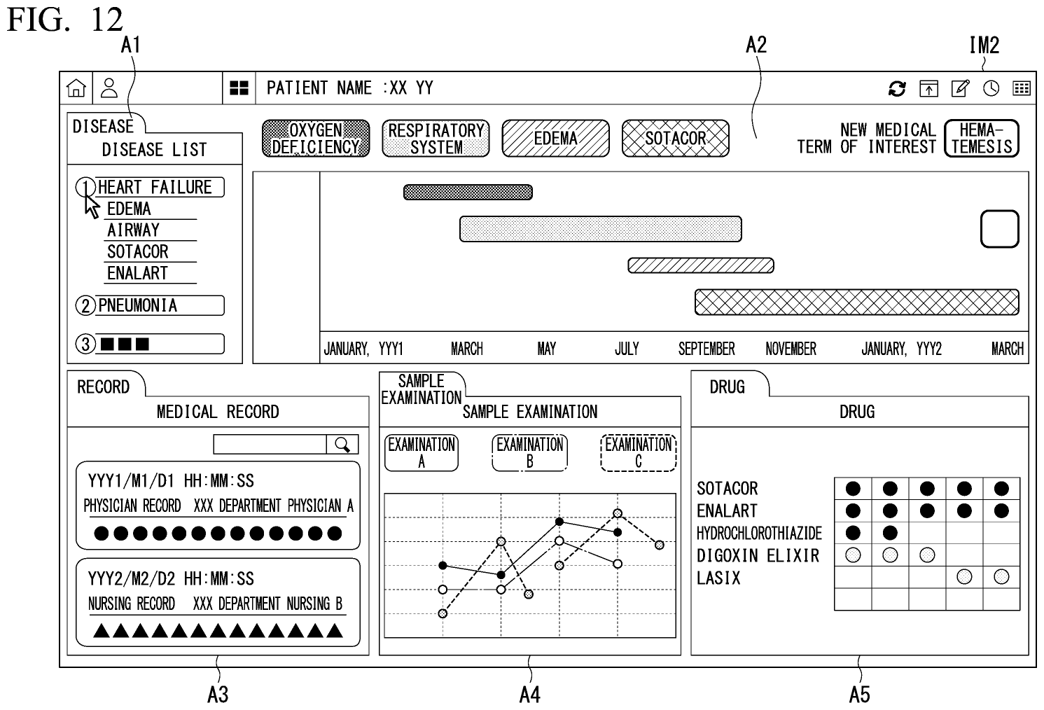

FIG. 12 is a diagram showing an example of a display screen on which the medical information processing device 100 according to the embodiment provides information. In FIG. 12, an example in which a display screen IM2 in which information about the medical term “hematemesis,” which is a medical term of interest having high importance, is added to the display screen IM shown in FIG. 10 is displayed on the display device is shown. In the display screen IM2 shown in FIG. 12, the information presentation area A2 shows time information in which the medical term “hematemesis” is newly extracted. Through the display screen IM2, the primary care physician can confirm that a medical term (a new medical term of interest) that needs to be newly focused on has been extracted as information about the disease of the diagnosis target patient, in addition to the previous diagnosis result and the like.

Although an example of a case where information of a new medical term “hematemesis” of interest is presented (displayed) in the information presentation area A2 in the display screen IM2 shown in FIG. 12 is shown, this is only an example. A method in which the medical information processing device 100 provides the primary care physician with information of a new medical term of interest may be any method as long as it is a method of providing information so that the primary care physician can easily confirm the information. For example, the information provision function 160 may be performed to prominently present (display) a medical term that needs to be newly focused on in the information presentation areas A1 to A5 shown on the display screen IM2 or another information presentation area A.

### [Process of Medical Information Processing Device]

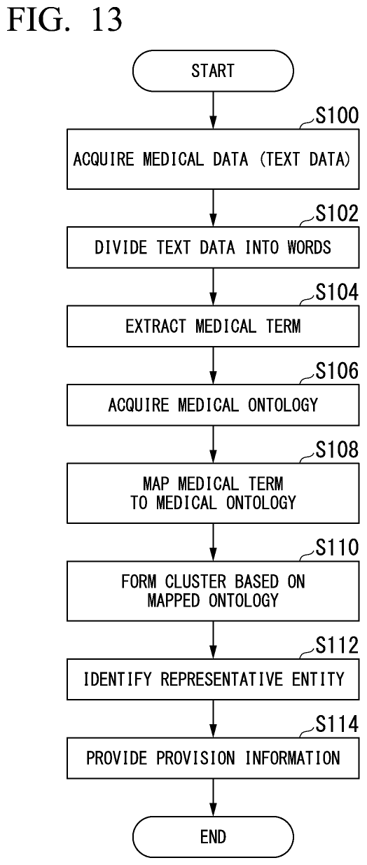

Next, an operation of the medical information processing device 100 will be described. FIG. 13 is a flowchart showing an example of a flow of a process of the medical information processing device 100 according to the embodiment. In FIG. 13, an example of a process in which the medical information processing device 100 filters and presents information about the disease of the diagnosis target patient is shown. For example, the medical information processing device 100 may be configured to present information about the disease of the diagnosis target patient in accordance with an instruction from the primary care physician when the diagnosis of the diagnosis target patient starts.

When an information presentation process is started in the medical information processing device 100 (the processing circuitry 110), the medical data acquisition function 120 is performed to acquire medical data (electronic medical records) of a diagnosis target patient stored in the medical data storage 10 (step S100). At this time, the medical data acquisition function 120 is performed to acquire text data of a text sentence included in the electronic medical record. The medical data acquisition function 120 is performed to output the acquired medical data (text data) to the medical data processing function 140.

The mapping processing function 142 of the medical data processing function 140 is performed to divide the text data acquired in the medical data acquisition function 120 into words (step S102). The mapping processing function 142 is performed to extract a medical term from each word obtained in the dividing process (step S104). Further, the mapping processing function 142 is performed to acquire (read) a medical ontology stored in the medical ontology storage 20 (step S106). Also, the mapping processing function 142 is performed to map the extracted medical term to the corresponding entity within the acquired medical ontology (step S108). That is, the mapping processing function 142 is performed to generate a mapped ontology. The mapping processing function 142 is performed to output the generated mapped ontology to the identification function 144.

The identification function 144 is performed to trace the adjacency of the entity to which the medical term is mapped on the basis of the mapped ontology output by the mapping processing function 142 and form a cluster in which a label indicating a prescribed connection frequency is connected to the target entity (step S110). Also, the identification function 144 is performed to identify an entity indicating medical information indicating a specific condition in the formed cluster as a representative entity (step S112). The identification function 144 is performed to output information indicating the identified representative entity and the formed cluster to the information provision function 160.

The information provision function 160 is performed to generate and provide provision information (for example, the display screen IM shown in FIG. 10) based on the information indicating the representative entity output in the identification function 144 and the formed cluster (step S114). Also, the medical information processing device 100 (the processing circuitry 110) ends the process of the present flowchart.

In this way, the medical information processing device 100 can organize the information included in the medical data (electronic medical records) of the diagnosis target patient stored in the medical data storage 10 on the basis of the medical ontology and provide the organized information so that the primary care physician for the diagnosis target patient can easily confirm the information.

As described above, in the medical information processing device of the embodiment, the information included in the text data of the medical data (electronic medical records) of the diagnosis target patient stored in the medical data storage 10 is divided into words and the medical term is extracted from each word obtained in the dividing process. Also, in the medical information processing device of the embodiment, a mapped ontology in which the extracted medical term is mapped to the corresponding entity in the medical ontology is generated. Thereby, in the medical information processing device of the embodiment, it is possible to organize the adjacency of medical terms described in the medical data (electronic medical records) of the diagnosis target patient. Also, in the medical information processing device of the embodiment, a representative entity representing the generated mapped ontology is identified. Thereby, in the medical information processing device of the embodiment, information about entities related to the identified representative entity (lower-level entities such as “symptom,” “site,” and “therapeutic drug”) can be provided so that the information is easily confirmed by the primary care physician for the diagnosis target patient. In other words, in the medical information processing device of the embodiment, medical term information focusing on the identified representative entity can be provided to the primary care physician so that the information is easily confirmed. Thereby, in a medical institution where the medical information processing device of the embodiment is introduced, the primary care physician can perform the appropriate diagnosis on the diagnosis target patient.

In the above-described embodiment, a case where the medical information processing device 100 identifies an entity (higher-level entity) indicating the name of a disease suffered by a diagnosis target patient as a representative entity has been described. However, the representative entity identified by the medical information processing device 100 may be an entity different from the entity indicating the name of the disease. For example, the medical information processing device 100 may provide a therapeutic drug corresponding to a disease suffered by a diagnosis target patient, a therapeutic method for a disease suffered by the diagnosis target patient, or the like. An entity serving as a representative entity in the medical information processing device 100 may be designated by, for example, the primary care physician. In this case, the designation of the entity as a representative entity by the primary care physician, for example, may be performed on the display screen IM shown in FIG. 10 when the primary care physician operates the input interface (not shown) provided by the medical information processing device 100. In this case, it is only necessary for the functional configuration, operation, process, and the like of the medical information processing device 100 to be equivalent to the functional configuration, operation, process, and the like of the medical information processing device 100 of the above-described embodiment and these can be easily conceivable. Therefore, a detailed description of the functional configuration, operation, process, and the like of the medical information processing device 100 in this case will be omitted.

Although the timing when the mapping processing function 142 generates a mapped ontology is not particularly described in the above-described embodiment, the timing when the mapped ontology is generated may be any timing as long as it is a timing when new information is added to the medical data (electronic medical records) of the diagnosis target patient. This is because the medical data (electronic medical records) of the diagnosed patient is considered unchanged until a new examination or diagnosis is performed even though a mapped ontology is generated at any timing after the results of examinations and diagnoses performed on the diagnosis target patient are updated. For this reason, the mapping processing function 142 may be performed to generate a mapped ontology when the medical data (electronic medical records) is updated and cause the generated mapped ontology to be stored in the storage device (not shown) or the like. In this case, the medical information processing device 100 can perform a process from an identification process of the identification function 144 for the mapped ontology stored in the storage device (not shown) or the like. That is, even if the medical information processing device 100 provides the same provision information to the primary care physician, it is possible to distribute a processing load in the medical information processing device 100 and reduce the load of processes to be simultaneously performed to provide the provision information by storing the generated mapped ontology in the storage device (not shown) or the like. In this case, because the functional configuration, operation, process, and the like of the medical information processing device 100 can be easily conceived on the basis of the functional configuration, operation, processing, and the like of the medical information processing device 100 of the above-described embodiment, detailed description thereof is omitted.

The above-described embodiment can be represented as follows.

A medical information processing device including:
processing circuitry,
wherein the processing circuitry
acquires text data of a processing target; and
outputs specific information for identifying one item of medical information satisfying a specific condition regarding a diagnosis target on the basis of a medical term included in the text data and a medical ontology in which a relationship between two or more items of the medical information is defined.

According to at least one embodiment described above, there is provided processing circuitry (120 or 140) configured to acquire text data of a processing target (medical data); and output specific information (a representative entity) for identifying one item of medical information satisfying a specific condition regarding a diagnosis target (a diagnosis target patient) on the basis of a medical term included in the text data and a medical ontology in which a relationship between two or more items of the medical information (higher-level entity+lower-level entities) is defined, whereby information included in the medical data can be organized.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing device comprising:
processing circuitry configured to
acquire text data of a processing target; and
output specific information for identifying one item of medical information satisfying a specific condition regarding a diagnosis target based on a medical term included in the text data and a medical ontology in which a relationship between two or more items of the medical information is defined.

2. The medical information processing device according to claim 1, wherein the processing circuitry
generates a modified medical ontology in which the medical term is mapped to corresponding medical information defined in the medical ontology and
identifies the specific information based on the modified medical ontology.

3. The medical information processing device according to claim 2, wherein the processing circuitry identifies the specific information based on a connection frequency of second medical information that is the medical information to which the medical term is mapped for first medical information that is the medical information to which the medical term is not mapped in the modified medical ontology.

4. The medical information processing device according to claim 3,
wherein the medical information is classified into at least one category, and
wherein the processing circuitry identifies the specific information based on the connection frequency of the second medical information for the first medical information belonging to at least a common category.

5. The medical information processing device according to claim 3, wherein the processing circuitry identifies the specific information based on the connection frequency of the second medical information for the first medical information to which the second medical information is connected among a plurality of items of the first medical information having a common higher-level concept.

6. The medical information processing device according to claim 2,
wherein the medical information includes at least one feature term, and
wherein the processing circuitry identifies the specific information based on a connection frequency based on the feature term of second medical information that is the medical information to which the medical term is mapped for first medical information that is the medical information to which the medical term is not mapped in the modified medical ontology.

7. The medical information processing device according to claim 2, wherein the processing circuitry
obtains a co-occurrence degree of the medical term included in the text data, and
identifies the specific information based on a connection frequency based on the co-occurrence degree of second medical information that is the medical information to which the medical term is mapped for first medical information that is the medical information to which the medical term is not mapped in the modified medical ontology.

8. The medical information processing device according to claim 7, wherein the processing circuitry identifies the specific information based on the connection frequency including a new medical term without the co-occurrence degree included in the text data.

9. The medical information processing device according to claim 2, wherein the processing circuitry
obtains a probability distribution in which each medical term appears in the text data, and
generates a modified medical ontology to which the medical term is mapped based on the probability distribution.

10. The medical information processing device according to claim 2, wherein the processing circuitry generates the modified medical ontology by inputting the medical term to a trained model trained using the medical ontology as a graph structure.

11. The medical information processing device according to claim 1, wherein the processing circuitry causes a display device to display display content indicating the medical information identified by the specific information.

12. The medical information processing device according to claim 11, wherein the display content newly satisfies the specific condition regarding the diagnosis target and includes one item of the medical information of interest.

13. The medical information processing device according to claim 1, wherein the specific condition is used to identify the medical information about a disease of the diagnosis target.

14. The medical information processing device according to claim 1, wherein the specific condition is used to identify the medical information about a therapeutic method for a disease of the diagnosis target.

15. A medical information processing method comprising:
acquiring, by a computer, text data of a processing target; and
outputting, by the computer, specific information for identifying one item of medical information satisfying a specific condition regarding a diagnosis target based on a medical term included in the text data and a medical ontology in which a relationship between two or more items of the medical information is defined.

16. A non-transitory computer-readable storage medium storing a program for causing a computer to:
acquire text data of a processing target; and
output specific information for identifying one item of medical information satisfying a specific condition regarding a diagnosis target based on a medical term included in the text data and a medical ontology in which a relationship between two or more items of the medical information is defined.
